(19) United States
(12) Patent Application Publication
(10) Pub. No.: US 2007/0233217 A1
(43) Pub. Date: Oct. 4, 2007

(54) Implantable medical electrode

(76) Inventors: Zhongping Yang, Woodbury, MN (US); Rick D. Mcvenes, Isanti, MN (US)

Correspondence Address: MEDTRONIC, INC., 710 MEDTRONIC PARKWAY NE, MINNEAPOLIS, MN, 55432-9924, US

(21) Appl. No.: 11/395,402

(22) Filed: Mar. 31, 2006

(51) Int. Cl. A61N 1/05

(52) U.S. Cl. 607/126

(57) ABSTRACT

An implantable medical electrode for use with implantable medical device systems having a base formed of a tungsten alloy. The tungsten alloy electrode may be a lead-based or leadless electrode and may be provided with or without a coating. The tungsten alloy electrode is utilized in an implantable medical device that includes an electrode having a base formed of a tungsten alloy, an electrical contact, and an electronics module adapted to be electrically coupled to the electrode via the electrical contact.

### TECHNICAL FIELD

The invention relates generally to implantable medical devices and, in particular, to implantable medical electrodes formed from a tungsten alloy.

### BACKGROUND

Medical electrical leads are used in conjunction with a variety of electronic implantable medical devices such as pacemakers, cardioverter defibrillators, neurostimulators, and ECG monitors. The medical leads carry one or more electrodes used for sensing electrical signals in the body, such as intracardiac electrogram (EGM) signals, electrocardiogram (ECG) signals, and electromyogram (EGM) signals. Electrodes are also used for delivering therapeutic electrical stimulation pulses or for delivering electrical pulses used in electrophysiological mapping or for other diagnostic purposes.

In selecting materials for fabricating a medical electrode, considerations include the biocompatibility, electrical properties, mechanical properties, chemical stability, the radiographic visibility of the material and the electrode-tissue interfacial impedance. Known or proposed medical electrodes are fabricated with a base material formed from platinum, titanium, tantalum, stainless steel, iridium, or alloys thereof. Platinum and platinum-iridium provide good electrical and mechanical properties, are chronically biostable and are highly visible under radiography. For these reasons, platinum and platinum-iridium, though relatively costly materials, are commonly used for manufacturing medical electrodes intended for chronic implantation. The base electrode material is often coated with a low polarization coating to reduce the effects of polarization at the tissue-electrode interface, which can interfere with electrode performance. Known or proposed medical electrode coatings include platinum black and porous carbide, nitride, carbonitride or oxide layers formed form titanium, vanadium zirconium, niobium, molybdenum, hafnium, tantalum, iridium, platinum, and tungsten.

### BRIEF DESCRIPTION OF THE DRAWINGS

FIG. 1 is a plan view of one embodiment of a medical electrical lead.

FIG. 2 is a sectional view of the distal end of an electrical medical lead provided with an active fixation electrode.

FIG. 3 is a sectional view of a tungsten alloy electrode provided with a coating.

FIG. 4 is a schematic diagram of an IMD and associated leads carrying tungsten alloy electrodes implanted in relation to a patient's heart.

FIG. 5 is a top and plan view of an IMD incorporating tungsten alloy electrodes disposed along the IMD housing for subcutaneously sensing and/or delivering electrical stimulation pulses.

FIG. 6 is a flow chart summarizing a method for manufacturing an implantable medical electrode using a tungsten alloy base material.

### DETAILED DESCRIPTION

In the following description, references are made to illustrative embodiments for carrying out the invention. It is understood that other embodiments may be utilized without departing from the scope of the invention. For purposes of clarity, the same reference numbers are used in the drawings to identify similar elements. Unless otherwise noted, drawing elements are not shown to scale.

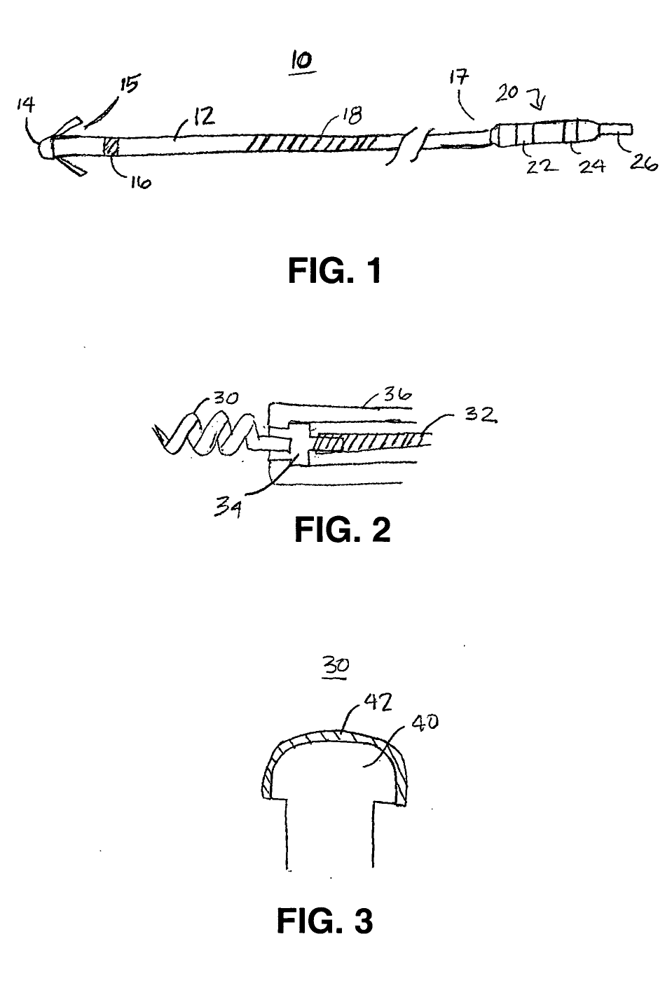

FIG. 1 is a plan view of one embodiment of a medical electrical lead. Lead 10 includes an elongated lead body 12 extending between a distal end 15 and a proximal end 17. A tip electrode 14 is provided at distal lead end 15. A ring electrode 16 is spaced proximally from tip electrode 14 and a coil electrode 18 is spaced proximally from ring electrode 16. Each electrode 14, 16 and 18 is individually coupled to an insulated conductor extending through lead body 12 to a connector 22, 24 or 26 included in proximal connector assembly 20. Proximal connector assembly 20 is adapted to be inserted in a connector bore provided in an implantable medical device for electrically connecting electrodes 14, 16 and 18 to electronics included in the IMD.

Any of tip electrode 14, ring electrode 16, and coil electrode 18 are formed having an electrode base fabricated from a tungsten alloy. Tungsten has good electrical properties for use as an implantable electrode, is radiographically visible and is relatively low in cost. However, tungsten has not been used commercially as a base material for chronically implanted medical electrodes because it is not biostable in the implanted environment and will degrade over time. As used herein, “chronic” refers to implant durations exceeding about 24 hours with the expectation that the device will generally remain implanted for days, weeks, months or years. IMDs implanted acutely may be implanted for a few minutes or hours and are generally used for diagnostic testing or performing a surgical or other clinical procedure, such as electrophysiological mapping, tissue ablation, angioplasty, imaging or other procedures.

It is desirable that a tungsten alloy used for manufacturing implantable electrodes provide the chemical stability needed for chronic implantation Other properties of tungsten include low cost, radiographic visibility, and high thermal conductivity. The high thermal conductivity of tungsten may act to prevent tissue heating at the electrode tissue interface during MRI procedures. Although tungsten alloy electrodes are expected to have the chemical stability needed for chronic implantation, embodiments of the present invention are not limited to electrodes intended for chronic use only but may also include electrodes implanted acutely.

The mechanical properties of tungsten, such as durability and machinability, can be improved by combining tungsten with one or more other metals to form an alloy. The tungsten alloy can be used to form any electrode base configuration, including, but not limited to, tip electrodes such as the electrode 14 shown in FIG. 1, button electrodes, ring electrodes, coil electrodes, patch electrodes, as well as active fixation electrodes such as helical electrodes and “fish hook” electrodes. A tungsten alloy electrode may be manufactured using machining processes applied to a solid piece of material, sintering a powdered form of a tungsten alloy, casting, or other appropriate methods.

FIG. 2 is a sectional view of the distal end of an electrical medical lead provided with an active fixation electrode. Fixation electrode 30 is embodied as a helical electrode and is electrically coupled to a conductor 32 via sleeve 34. Lead-based electrodes such as fixation electrode 30 and any of the electrodes 14, 16, and 18 shown in FIG. 1 are typically electrically coupled to a welding or crimping sleeve, which is further coupled to a conductor. Conductor 32 may be provided as any wire, stranded or multifilar, coiled, or cable type conductor. Conductor 32 is coupled to sleeve 32 using any appropriate method such as welding, staking, crimping or riveting. Conductor 32 extends through lead body 36 to a connector assembly at a proximal lead body end.

FIG. 3 is a sectional view of a tungsten alloy electrode provided with a coating. Electrode 38 includes electrode base 40 formed from a tungsten alloy and a low-polarization coating 42 applied to base 40 for enhancing the electrical performance of electrode 38. In various embodiments, the tungsten alloy used to form base 40 includes one or more other metals, such as, but not limited to, titanium, tantalum, iridium, niobium, platinum, zirconium, hafnium, nickel, iron, molybdenum, and vanadium. A tungsten alloy used to form base 40 is generally composed of at least about 50% tungsten to take advantage of the properties of tungsten such as the lower cost and radiographic visibility, although compositions having less than 50% tungsten are not beyond the scope of the invention. In one embodiment, base 40 is formed from about 50% tungsten and about 50% titanium. In another embodiment, base 40 is formed from about 97% tungsten, about 2% nickel and about 1% iron.

Base 40 may be manufactured using methods, such as sintering or machining processes, that result in a porous or other structured surface for increasing the surface area of base 40. Alternatively, base 40 may undergo surface-enhancing treatments, such as mechanical etching, prior to applying low-polarization coating 42. Low polarization coating 42 may be applied by sputtering, dipping, chemical vapor deposition or other appropriate method depending on the type of coating being applied. Low polarization coating 42 may be formed from platinum black or a porous nitride, carbide, carbonitride or oxide layer of titanium, vanadium, zirconium, niobium, molybdenum, hafnium, tantalum, iridium, platinum, and tungsten.

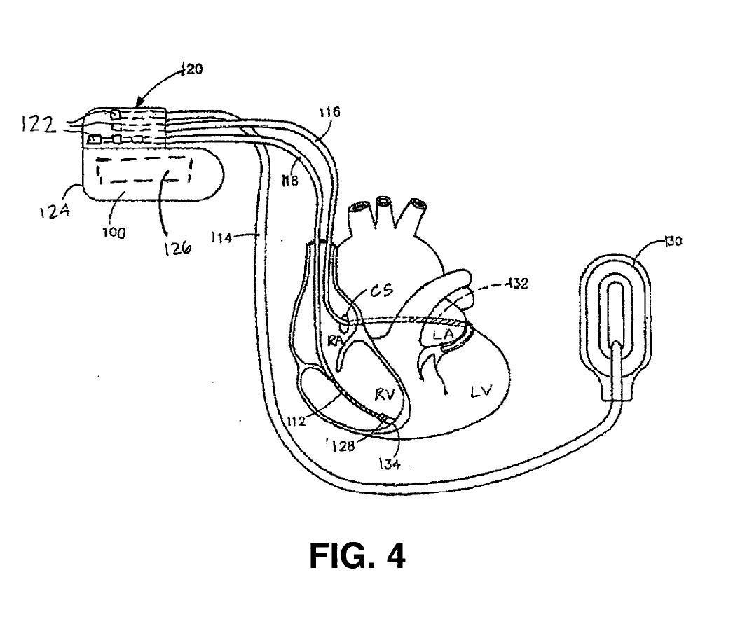

FIG. 4 is a schematic diagram of an IMD and associated leads carrying tungsten alloy electrodes implanted in relation to a patient's heart. IMD 100 is embodied as an implantable cardioverter defibrillator (ICD) providing sensing of EGM signals and delivering therapeutic electrical stimulation pulses for pacing, cardioverting, and defibrillating the heart as needed. IMD 100 includes a hermetically sealed housing 124 for enclosing an electronics module 126 therein. As used herein, the term “module” refers to an application specific integrated circuit (ASIC), an electronic circuit, a processor (shared, dedicated, or group) and memory that execute one or more software or firmware programs, a combinational logic circuit, or other suitable components that provide the described functionality. IMD 100 generally includes a low voltage power source for powering electronics module 126 and one or more high energy density capacitors for generating high voltage cardioversion and defibrillation shocking pulses.

IMD 100 is provided with a connector block 120 formed with one or more connector bores for receiving each of the associated leads 114, 116, and 118 used with IMD 100. Connector block 120 includes electrical contacts 122 which mate with connectors included on proximal connector assemblies included on leads 114, 116, and 118. Electrical contacts 122 are electrically coupled to electronics module 126 via insulated feedthrough conductors extending through IMD housing 124. In this way, various electrodes carried by leads 114, 116, and 118, including tip electrode 134, ring electrode 128, coil electrode 112, coil electrode 132 and subcutaneous patch electrode 130, are electrically coupled to IMD electronics module 126 for carrying out sensing and stimulation functions. Any of the electrodes shown, 112, 128, 130, 132, and 134 may be formed with a tungsten alloy base material.

While a particular IMD is shown associated with cardiac leads adapted to deploy electrodes 112, 128, 130, 132, and 134 in operative relation to the heart, it is recognized that any electrodes carried by leads associated with an IMD, including any pacemakers, ICDs, cardiac or other physiologic monitors, and neurostimulators, may be formed with a tungsten alloy base material.

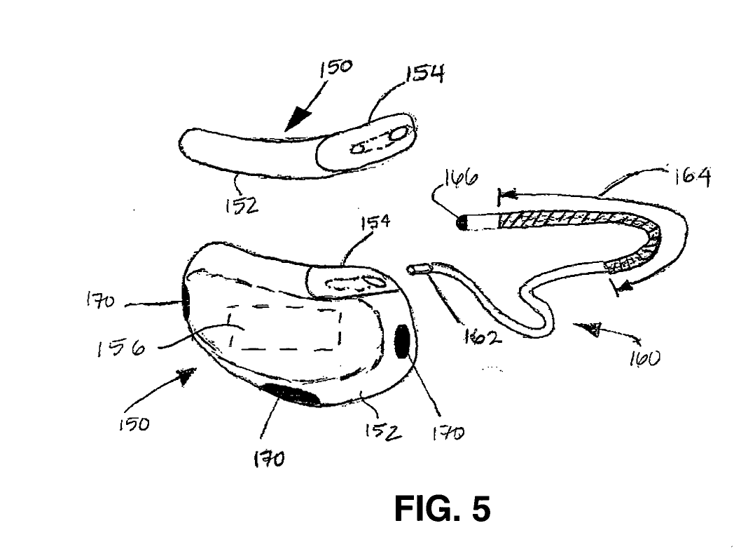

FIG. 5 is a top and plan view of an IMD incorporating tungsten alloy electrodes disposed along the IMD housing for subcutaneously sensing and/or delivering electrical stimulation pulses. Embodiments of the present invention include both lead-based and leadless electrodes. IMD 150 is embodied as a subcutaneous ICD. IMD 150 includes a generally ovoid housing 152 having a substantially kidney-shaped profile. Connector block 154 is coupled to housing 152 for receiving the connector assembly 162 of subcutaneous lead 160. IMD housing 150 is hermetically sealed and may be constructed of stainless steel, titanium or ceramic. Electronics module 156 enclosed in housing 152 may be incorporated on a polyamide flex circuit, printed circuit board (PCB) or ceramic substrate with integrated circuits packaged in leadless chip carriers and/or chip scale packaging (CSP). The plan view shows the generally ovoid construction of housing 152 that promotes ease of subcutaneous implant. This structure is ergonomically adapted to minimize patient discomfort during normal body movement and flexing of the thoracic musculature.

Subcutaneous lead 160 includes distal coil electrode 164, distal sensing electrode 166, an insulated flexible lead body and a proximal connector assembly 162 adapted for connection to IMD 150 via connector block 154. IMD 150 is provided with one or more housing-based electrodes forming a subcutaneous electrode array (SEA) 170. Three electrodes positioned in an orthogonal arrangement are included in SEA 170 in the embodiment shown in FIG. 5. Other embodiments of an IMD incorporating leadless electrodes may include any number of electrodes mounted on or incorporated in housing 152. Multiple subcutaneous electrodes are provided to allow multiple sensing vector configurations.

Electrode assemblies included in SEA 170 are welded into place on the flattened periphery of housing 152. The complete periphery of IMD 150 may be manufactured to have a slightly flattened perspective with rounded edges to accommodate the placement of SEA assemblies. The SEA electrode assemblies are welded to housing 152 (in a manner that preserves hermaticity of the housing 152) and are connected via conductors (not shown in FIG. 5) to internal electronics module 156. SEA 170 may be constructed using tungsten alloy electrodes in the form of flat plates, or alternatively, spiral electrodes. SEA 170 may be mounted in a non-conductive surround shroud Examples of electrode assemblies that may be used for constructing SEA 170 are generally described in U.S. Pat. No. 6,512,940 (Brabec, et al.), U.S. Pat. No. 6,522,915 (Ceballos, et al.) or in U.S. Pat. No. 6,622,046 (Fraley, et al.), all of which patents are hereby incorporated herein by reference in their entireties.

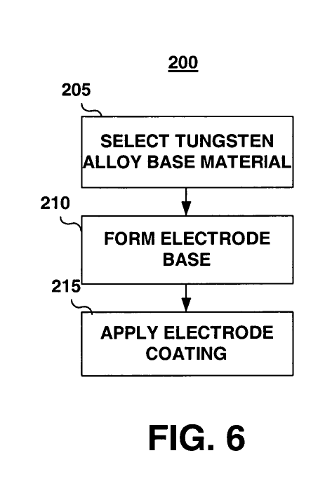

FIG. 6 is a flow chart summarizing a method for manufacturing an implantable medical electrode using a tungsten alloy base material. At block 205, a tungsten alloy base material is selected and formed into an electrode base at block 210. The electrode base may be formed into any type of implantable electrode, including those described above. A coating is optionally applied at block 215. Coatings that may be applied to a tungsten alloy electrode include low polarization coatings or other coatings used to improve the performance of the electrode acutely and/or chronically. The electrode may then be assembled on a lead or an IMD housing for use in an IMD system.

Thus, tungsten alloy electrodes for use in implantable medical device systems have been presented in the foregoing description with reference to specific embodiments. It is appreciated that various modifications to the referenced embodiments may be made without departing from the scope of the invention as set forth in the following claims.

1. An implantable medical device system, comprising:
an electrode having a base formed of a tungsten alloy;
an electrical contact; and
an electronics module adapted to be electrically coupled to the electrode via the electrical contact.

2. The device of claim 1 further comprising:
an elongated insulative lead body, wherein the electrode is disposed along the lead body; and
a conductor coupled to the electrode and extending through the elongated lead body, the conductor being adapted to be electrically coupled to the electrical contact.

3. The device of claim 2 wherein the electrode is one of an active fixation electrode, a ring electrode, a coil electrode, a patch electrode and a tip electrode.

4. The device of claim 1 further comprising a medical device housing and wherein the electrode is disposed along the medical device housing.

5. The device of claim 4 wherein the electrode is one of a flat plate electrode and a spiral electrode.

6. The device of claim 1 further comprising a coating applied over the electrode base.

7. The device of claim 1 wherein the tungsten alloy includes at least one of platinum, tantalum, titanium, nickel, iron, iridium, zirconium, niobium, and vanadium.

8. The device of claim 1 wherein the electrode base includes at least approximately fifty percent tungsten.

9. A medical electrical lead, comprising:
an elongated lead body;
an electrode disposed along the lead body having a base formed of a tungsten alloy; and
a conductor electrically coupled to the electrode and extending through the lead body.

10. The lead of claim 9 wherein the electrode is one of an active fixation electrode, a ring electrode, a coil electrode, a patch electrode, and a tip electrode.

11. The lead of claim 9 further comprising a coating disposed over the electrode base.

12. The lead of claim 9 wherein the tungsten alloy includes at least one of platinum, tantalum, titanium, nickel, iron, iridium, zirconium, niobium, and vanadium.

13. The lead of claim 9 wherein the base includes at least about fifty percent tungsten.

14. A method for manufacturing a medical electrode for use in association with an implantable medical device, comprising:
forming an electrode base from a tungsten alloy, and
coating the base with a low polarization coating.

15. The method of claim 14 wherein the electrode is one of an active fixation electrode, a tip electrode, a ring electrode, a coil electrode, a flat plate electrode, and a spiral electrode.

16. The method of claim 14 further comprising disposing the electrode along an elongated lead body.

17. The method of claim 14 further comprising disposing the electrode along a housing of the implantable medical device.

18. The method of claim 14 wherein the tungsten alloy includes one of platinum, tantalum, titanium, nickel, iron, iridium, zirconium, niobium, and vanadium.

19. The method of claim 14 wherein the base includes at least approximately fifty percent tungsten.

20. The method of claim 14 wherein the coating is applied using chemical vapor deposition.
